(19) United States
(12) Patent Application Publication
(10) Pub. No.: US 2014/0121450 A1
(43) Pub. Date: May 1, 2014

(54) Magnetic Abutment Systems, Devices, Components and Methods for Bone Conduction Hearing Aids

(71) Applicant: Sophono, Inc., (US)

(72) Inventors: James F. Kasic, Boulder, CO (US); Nicholas F. Pergola, Arvada, CO (US); Markus C. Haller, Gland (CH)

(73) Assignee: Sophono, Inc., Boulder, CO (US)

(21) Appl. No.: 13/650,026

(22) Filed: Oct. 11, 2012

Related U.S. Application Data
(63) Continuation-in-part of application No. 13/550,581, filed on Jul. 16, 2012.

(51) Int. Cl. A61F 11/04; H04R 25/00

(52) U.S. Cl. 600/25

(57) ABSTRACT

Various embodiments of systems, devices, components, and methods are disclosed for magnetically coupling ferrous or magnetic spacers of bone conduction hearing aids to ferrous or magnetic bone screws or implants affixed to a patient's skull. In one embodiment, a spacer is provided that is configured to magnetically couple the hearing aid to an abutment and corresponding bone screw, or to an implanted ferrous or magnetic member, that is affixed to the patient's skull.

### RELATED APPLICATIONS

This application is a continuation-in-part of, and claims priority and other benefits from, U.S. patent application Ser. No. 13/550,581 entitled “Systems, Devices, Components and Methods for Bone Conduction Hearing Aids” to Pergola et al. filed Jul. 16, 2012 (hereafter “the '581 patent application”). The '581 patent application is hereby incorporated by reference herein, in its entirety.

This application also hereby incorporates by reference, each in its respective entirety, the following patent applications filed on even date herewith: (1) U.S. patent application Ser. No. ______ entitled “Adjustable Magnetic Systems, Devices, Components and Methods for Bone Conduction Hearing Aids” to Kasic et al. having Attorney Docket No. P SPH 101; (2) U.S. patent application Ser. No. ______ entitled “Magnetic Spacer Systems, Devices, Components and Methods for Bone Conduction Hearing Aids” to Kasic et al. having Attorney Docket No. P SPH 105, and (3) U.S. patent application Ser. No. ______ entitled “Abutment Attachment Systems, Mechanisms, Devices, Components and Methods for Bone Conduction Hearing Aids” to Kasic et al. having Attorney Docket No. P SPH 110.

### FIELD OF THE INVENTION

Various embodiments of the invention described herein relate to the field of systems, devices, components, and methods for bone conduction hearing aid devices.

### BACKGROUND

A bone-anchored hearing device (or “BAHD”) is an auditory prosthetic device based on bone conduction having a portion or portions thereof which are surgically implanted. A BAHD uses the bones of the skull as pathways for sound to travel to a patient's inner ear. For people with conductive hearing loss, a BAHD bypasses the external auditory canal and middle ear, and stimulates the still-functioning cochlea via an implanted metal post. For patients with unilateral hearing loss, a BAHD uses the skull to conduct the sound from the deaf side to the side with the functioning cochlea. In most BAHA systems, a titanium post or plate is surgically embedded into the skull with a small abutment extending through and exposed outside the patient's skin. A BAHD sound processor attaches to the abutment and transmits sound vibrations through the external abutment to the implant. The implant vibrates the skull and inner ear, which stimulates the nerve fibers of the inner ear, allowing hearing. A BAHD device can also be connected to an FM system or iPod by means of attaching a miniaturized FM receiver or Bluetooth connection thereto.

BAHD devices manufactured by COCHLEAR™ of Sydney, Australia, and OPTICON™ of Smoerum, Sweden. SOPHONO™ of Boulder, Colo. manufactures a an ALPHA 1 magnetic hearing aid device, which attaches by magnetic means behind a patient's ear to the patient's skull by coupling to a magnetic or magnetized bone plate (or “magnetic implant”) implanted in the patient's skull beneath the skin.

Surgical procedures for implanting such posts or plates are relatively straightforward, and are well known to those skilled in the art. See, for example, “Alpha I (S) & Alpha I (M) Physician Manual—REV A S0300-00” published by Sophono, Inc. of Boulder, Colo., the entirety of which is hereby incorporated by reference herein.

Hearing aid devices and systems offered by different manufacturers are often incompatible with one another such that external hearing aids provided by one manufacturer cannot be used in conjunction with bone screws or magnetic implants provided by another manufacturer. This results in patients and health care providers being unable to mix or combine, by way of example, hearing aids provided by one manufacturer with bone screws or magnetic implants provided by another manufacturer.

What is needed is the ability of patients and health care providers to employ hearing aid system components or devices provided by one manufacturer with those of another manufacturer.

### SUMMARY

In one embodiment, there is provided a magnetic hearing aid system comprising a bone conduction hearing aid comprising an electromagnetic (“EM”) transducer and a spacer comprising at least a first magnetic or ferrous member, the spacer being configured to be mechanically and acoustically coupled to the EM transducer, and to be magnetically and acoustically coupled to an external hearing aid abutment attached to or forming an external portion of a bone screw implanted in a patient's skull, and at least a second magnetic or ferrous member configured to be mounted on or attached to the external hearing aid abutment, wherein when the second magnetic or ferrous member is mounted on or attached to the external hearing aid abutment, and the first magnetic or ferrous member of the spacer is placed over and in operable proximity to the mounted or attached second magnetic or ferrous member, the first magnetic or ferrous member and the second magnetic or ferrous member magnetically couple to one another with sufficient magnetic coupling force to secure the hearing aid to the second magnetic or ferrous member and the external hearing aid abutment, and further wherein at least one of the first magnetic or ferrous member and the second magnetic or ferrous member is magnetic.

In another embodiment, there is provided a second magnetic or ferrous member configured to be mounted on or attached to an external hearing aid abutment, the hearing aid abutment being configured to be attached to or form an external portion of a bone screw implanted in a patient's skull, the bone screw and hearing aid abutment being configured to operate in conjunction with a bone conduction hearing aid comprising an EM transducer and a spacer comprising at least a first magnetic or ferrous member, the spacer being configured to be mechanically and acoustically coupled to the EM transducer, and to be magnetically and acoustically coupled to the second magnetic or ferrous member and the external hearing aid abutment, wherein when the second magnetic or ferrous member is mounted on or attached to the external hearing aid abutment, and the first magnetic or ferrous of the spacer and hearing aid is placed over and in operable proximity to the mounted or attached second magnetic or ferrous member, the first magnetic or ferrous member and the second magnetic or ferrous member magnetically couple to one another with sufficient magnetic coupling force to secure the hearing aid to the second magnetic or ferrous member and the external hearing aid abutment, and further wherein at least one of the first magnetic or ferrous member and the second magnetic or ferrous member is magnetic.

In yet another embodiment, there is provided a method of magnetically coupling a first magnetic or ferrous member to a second magnetic or ferrous member, the second magnetic or ferrous member being configured to be mounted on or attached to an external hearing aid abutment, the hearing aid abutment being configured to be attached to or form an external portion of a bone screw implanted in a patient's skull, the bone screw and hearing aid abutment being configured to operate in conjunction with a bone conduction hearing aid comprising an EM transducer and a spacer comprising at least the first magnetic or ferrous member, the spacer being configured to be mechanically and acoustically coupled to the EM transducer, and to be magnetically and acoustically coupled to the second magnetic or ferrous member and the external hearing aid abutment, at least one of the first magnetic or ferrous member and the second magnetic or ferrous member being magnetic, the method comprising a patient or healthcare provider placing the first magnetic or ferrous member of the spacer and hearing aid over and in operable proximity to the mounted or attached second magnetic or ferrous member, and magnetically coupling the first magnetic or ferrous member to the second magnetic or ferrous member with sufficient magnetic coupling force to secure the hearing aid to the patient's skull.

In still another embodiment, there is provided a magnetic hearing aid system comprising a bone conduction hearing aid comprising an EM transducer and an attachment member configured to be mechanically and acoustically coupled to the EM transducer, a spacer comprising at least a first magnetic or ferrous member and an abutment shaped and configured to be mechanically and acoustically coupled to the attachment member, and a magnetic or ferrous implant comprising at least a second magnetic or ferrous member, the magnetic or ferrous implant being configured to be implanted beneath a patient's skin and affixed or attached to the patient's skull, wherein the first magnetic or ferrous member of the spacer is configured to be magnetically coupled to the second magnetic or ferrous member of the magnetic or ferrous implant through the patient's skin such that the hearing aid and the magnetic spacer may be operably secured to the patient's skull, and further wherein at least one of the first magnetic or ferrous member and the second magnetic or ferrous member is magnetic.

In a further embodiment, there is provided a spacer configured for use in a hearing aid system comprising a bone conduction hearing aid comprising an EM transducer, an attachment member configured to be mechanically and acoustically coupled to the EM transducer, and a magnetic or ferrous implant comprising at least a second magnetic or ferrous member, the magnetic or ferrous implant being configured to be implanted beneath a patient's skin and affixed or attached to the patient's skull, wherein the spacer comprises at least a first magnetic or ferrous member and an abutment shaped and configured to be mechanically and acoustically coupled to the attachment member, the first magnetic or ferrous member of the spacer being configured to be magnetically coupled to the second magnetic or ferrous member of the magnetic or ferrous implant through the patient's skin such that the hearing aid and the spacer may be operably secured to the patient's skull, and further wherein at least one of the first magnetic or ferrous member and the second magnetic or ferrous member is magnetic.

In still another embodiment, there is provided a method of magnetically coupling a first magnetic or ferrous member to a second magnetic or ferrous member, the first magnetic or ferrous member forming a portion of a spacer comprising an abutment, the spacer being configured for use in a hearing aid system comprising a bone conduction hearing aid and a magnetic or ferrous implant comprising at least a second magnetic or ferrous member, the hearing aid comprising an EM transducer and an attachment member configured to be mechanically and acoustically coupled to the EM transducer and the abutment, the magnetic or ferrous implant being configured to be implanted beneath a patient's skin and affixed or attached to the patient's skull, at least one of the first magnetic or ferrous member and the second magnetic or ferrous member being magnetic, the method comprising a patient or healthcare provider placing the first magnetic or ferrous member of the spacer and hearing aid over and in operable proximity to the implanted second magnetic or ferrous member, and magnetically coupling the first magnetic or ferrous member to the second magnetic or ferrous member with sufficient magnetic coupling force to secure the hearing aid to the patient's skull.

Further embodiments are disclosed herein or will become apparent to those skilled in the art after having read and understood the specification and drawings hereof.

### BRIEF DESCRIPTION OF THE DRAWINGS

Different aspects of the various embodiments will become apparent from the following specification, drawings and claims in which:

FIGS. 1(a), 1(b) and 1(c) show side cross-sectional schematic views of selected embodiments of prior art SOPHONO ALPHA 1, BAHA and AUDIANT bone conduction hearing aids, respectively;

FIG. 2(a) shows one embodiment of a prior art functional electronic and electrical block diagram of hearing aid 10 shown in FIGS. 1(a) and 3(b);

FIG. 2(b) shows one embodiment of a prior art wiring diagram for a SOPHONO ALPHA 1 hearing aid manufactured using an SA3286 DSP;

FIG. 3(a) shows one embodiment of prior art magnetic implant 20 according to FIG. 1(a);

FIG. 3(b) shows one embodiment of a prior art SOPHONO® ALPHA 1® hearing aid 10;

FIGS. 4 and 5 show two different embodiments of hearing aid 10 and corresponding spacer 50 and bone screw 115;

FIGS. 6 through 12 show various embodiments of abutment 19 and bone screw 115, and of corresponding second magnetic or ferrous member 61/65;

FIG. 13 shows one embodiment of spacer 50 comprising first magnetic or ferrous member 55/58 configured to couple magnetically to second magnetic or ferrous member 61/65, and

FIGS. 14 through 16 show embodiments of spacers 50 configured to operate in conjunction with magnetic or ferrous implant 20.

The drawings are not necessarily to scale. Like numbers refer to like parts or steps throughout the drawings.

### DETAILED DESCRIPTIONS OF SOME EMBODIMENTS

Described herein are various embodiments of systems, devices, components and methods for bone conduction and/or bone-anchored hearing aids.

A bone-anchored hearing device (or “BAHD”) is an auditory prosthetic device based on bone conduction having a portion or portions thereof which are surgically implanted. A BAHD uses the bones of the skull as pathways for sound to travel to a patient's inner ear. For people with conductive hearing loss, a BAHD bypasses the external auditory canal and middle ear, and stimulates the still-functioning cochlea via an implanted metal post. For patients with unilateral hearing loss, a BAHD uses the skull to conduct the sound from the deaf side to the side with the functioning cochlea. In most BAHA systems, a titanium post or plate is surgically embedded into the skull with a small abutment extending through and exposed outside the patient's skin. A BAHD sound processor attaches to the abutment and transmits sound vibrations through the external abutment to the implant. The implant vibrates the skull and inner ear, which stimulates the nerve fibers of the inner ear, allowing hearing. A BAHD device can also be connected to an FM system or iPod by means of attaching a miniaturized FM receiver or Bluetooth connection thereto.

BAHD devices manufactured by COCHLEAR™ of Sydney, Australia, and OPTICON™ of Smoerum, Sweden. SOPHONO™ of Boulder, Colo. manufactures an Alpha 1 magnetic hearing aid device, which attaches by magnetic means behind a patient's ear to the patient's skull by coupling to a magnetic or magnetized bone plate (or “magnetic implant”) implanted in the patient's skull beneath the skin.

Surgical procedures for implanting such posts or plates are relatively straightforward, and are well known to those skilled in the art. See, for example, “Alpha I (S) & Alpha I (M) Physician Manual—REV A S0300-00” published by Sophono, Inc. of Boulder, Colo., the entirety of which is hereby incorporated by reference herein.

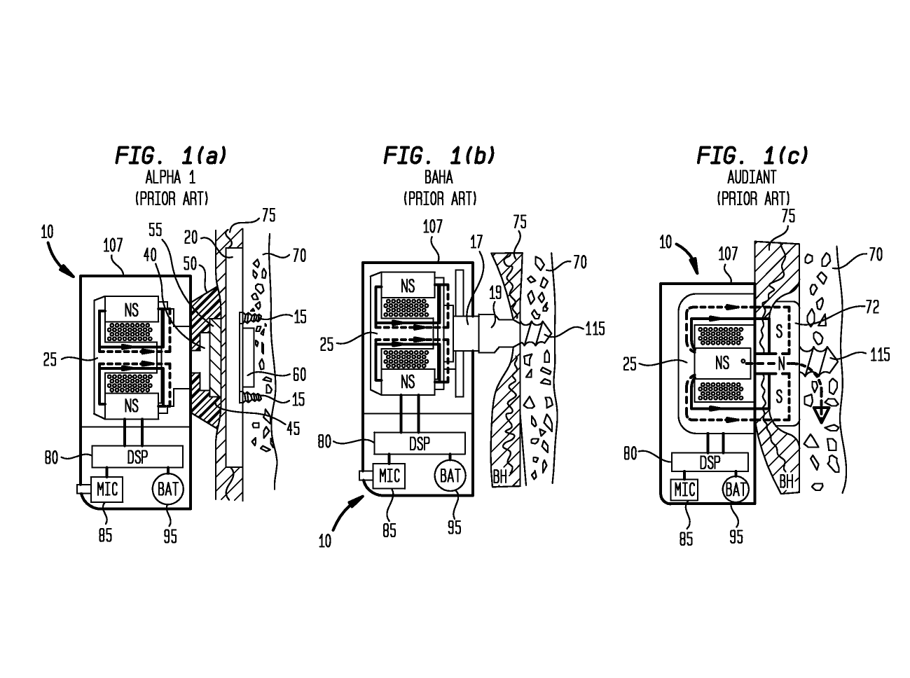

FIGS. 1(a), 1(b) and 1(c) show side cross-sectional schematic views of selected embodiments of prior art SOPHONO ALPHA 1, BAHA and AUDIANT bone conduction hearing aids, respectively. Note that FIGS. 1(a), 1(b) and 1(c) are not necessarily to scale.

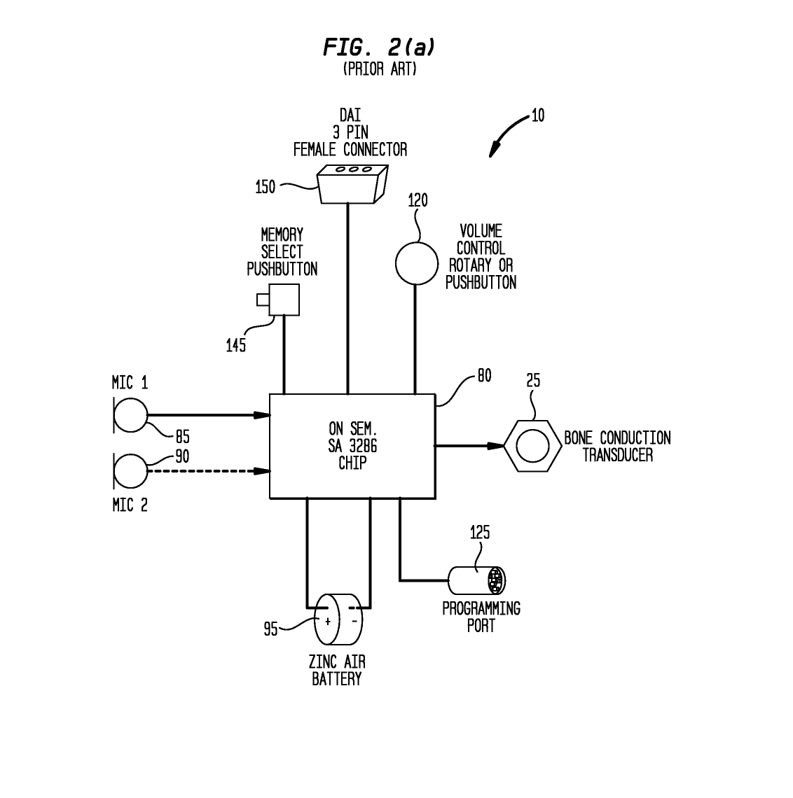

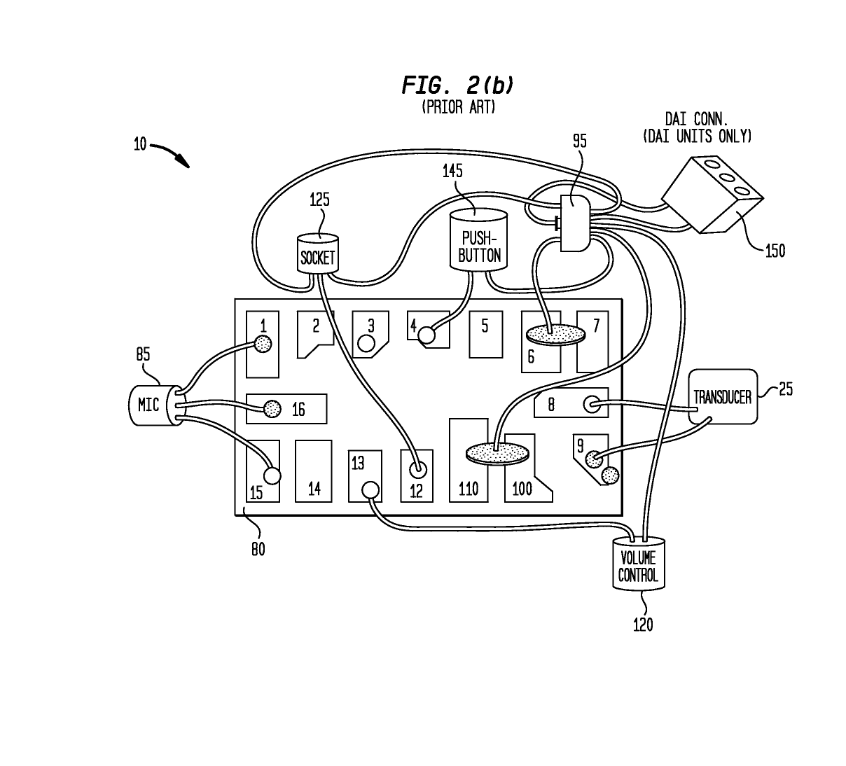

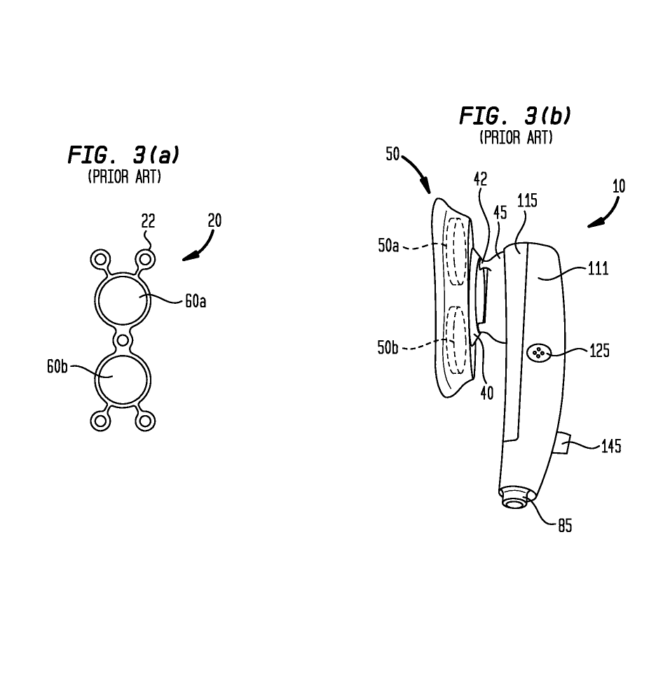

In FIG. 1(a), magnetic hearing aid device 10 comprises housing 107, electromagnetic/bone conduction (“EM”) transducer 25 with corresponding magnets and coils, digital signal processor (“DSP”) 80, battery 95, magnetic spacer 50, magnetic implant or magnetic implant bone plate 20. As shown in FIGS. 1(a) and 2(a), and according to one embodiment, magnetic implant 20 comprises a frame 21 (see FIG. 3(a)) formed of a biocompatible metal such as medical grade titanium that is configured to have disposed therein or have attached thereto implantable magnets or magnetic members 60. Bone screws 15 secure or affix magnetic implant 20 to skull 70, and are disposed through screw holes 22 of frame 21 (see FIG. 2(a)). Magnetic members 60 are configured to couple magnetically to one or more corresponding external magnetic members or magnets 55 mounted onto or into, or otherwise forming a portion of, magnetic spacer 50, which in turn is operably coupled to EM transducer 25 and metal disc 40. DSP 80 is configured to drive EM transducer 25, metal disk 40 and magnetic spacer 50 in accordance with external audio signals picked up by microphone 85. DSP 80 and EM transducer 25 are powered by battery 95, which according to one embodiment may be a zinc-air battery, or may be any other suitable type of primary or secondary (i.e., rechargeable) electrochemical cell such as an alkaline or lithium battery.

As further shown in FIG. 1(a), magnetic implant 20 is attached to patient's skull 70, and is separated from magnetic spacer 50 by patient's skin 75. Hearing aid device 10 of FIG. 1(a) is thereby operably coupled magnetically and mechanically to plate 20 implanted in patient's skull 70, which permits the transmission of audio signals originating in DSP 80 and EM transducer 25 to the patient's inner ear via skull 70.

FIG. 1(b) shows another embodiment of hearing aid 10, which is a BAHA® device comprising housing 107, EM transducer 25 with corresponding magnets and coils, DSP 80, battery 95, external post 17, internal bone anchor 115, and abutment member 19. In one embodiment, and as shown in FIG. 1(b), internal bone anchor 115 includes a bone screw formed of a biocompatible metal such as titanium that is configured to have disposed thereon or have attached thereto abutment member 19, which in turn may be configured to mate mechanically or magnetically with external post 17, which in turn is operably coupled to EM transducer 25. DSP 80 is configured to drive EM transducer 25 and external post 17 in accordance with external audio signals picked up by microphone 85. DSP 80 and EM transducer 25 are powered by battery 95, which according to one embodiment is a zinc-air battery (or any other suitable battery or electrochemical cell as described above). As shown in FIG. 1(b), implantable bone anchor 115 is attached to patient's skull 70, and is also attached to external post 17 through abutment member 19, either mechanically or by magnetic means. Hearing aid device 10 of FIG. 1(b) is thus coupled magnetically and/or mechanically to bone anchor 15 implanted in patient's skull 70, thereby permitting the transmission of audio signals originating in DSP 80 and EM transducer 25 to the patient's inner ear via skull 70.

FIG. 1(c) shows another embodiment of hearing aid 10, which is an AUDIANT®-type device, where an implantable magnetic member 72 is attached by means of bone anchor 115 to patient's skull 70. Internal bone anchor 115 includes a bone screw formed of a biocompatible metal such as titanium, and has disposed thereon or attached thereto implantable magnetic member 72, which couples magnetically through patient's skin 75 to EM transducer 25. DSP 80 is configured to drive EM transducer 25 in accordance with external audio signals picked up by microphone 85. Hearing aid device 10 of FIG. 1(c) is thus coupled magnetically to bone anchor 15 implanted in patient's skull 70, thereby permitting the transmission of audio signals originating in DSP 80 and EM transducer 25 to the patient's inner ear via skull 70.

FIG. 2(a) shows one embodiment of a prior art functional electronic and electrical block diagram of hearing aid 10 shown in FIGS. 1(a) and 2(b). In the block diagram of FIG. 2(a), and according to one embodiment, DSP 80 is a SOUND DESIGN TECHNOLOGIES® SA3286 INSPIRA EXTREME® DIGITAL DSP, for which data sheet 48550-2 dated March 2009, filed on even date herewith in an accompanying Information Disclosure Statement (“IDS”), is hereby incorporated by reference herein in its entirety. The audio processor for the SOPHONO ALPHA 1 hearing aid is centered around DSP chip 80, which provides programmable signal processing. The signal processing may be customized by computer software which communicates with the Alpha through programming port 125. According to one embodiment, the system is powered by a standard zinc air battery 95 (i.e. hearing aid battery), although other types of batteries may be employed. The SOPHONO ALPHA 1 hearing aid detects acoustic signals using a miniature microphone 85. A second microphone 90 may also be employed, as shown in FIG. 2(a). The SA 3286 chip supports directional audio processing with second microphone 90 to enable directional processing. Direct Audio Input (DAI) connector 150 allows connection of accessories which provide an audio signal in addition to or in lieu of the microphone signal. The most common usage of the DAI connector is FM systems. The FM receiver may be plugged into DAI connector 150. Such an FM transmitter can be worn, for example, by a teacher in a classroom to ensure the teacher is heard clearly by a student wearing hearing aid 10. Other DAI accessories include an adapter for a music player, a telecoil, or a Bluetooth phone accessory. According to one embodiment, DSP 80 or SA 3286 has 4 available program memories, allowing a hearing health professional to customize each of 4 programs for different listening situations. The Memory Select Pushbutton 145 allows the user to choose from the activated memories. This might include special frequency adjustments for noisy situations, or a program which is Directional, or a program which uses the DAI input.

FIG. 2(b) shows one embodiment of a prior art wiring diagram for a SOPHONO ALPHA 1 hearing aid manufactured using the foregoing SA3286 DSP. Note that the various embodiments of hearing aid 10 are not limited to the use of a SA3286 DSP, and that any other suitable CPU, processor, controller or computing device may be used. According to one embodiment, DSP 80 is mounted on a printed circuit board 155 disposed within housing 110 and/or housing 115 of hearing aid 10 (not shown in the Figures).

In some embodiments, the microphone incorporated into hearing aid 10 is an 8010T microphone manufactured by SONION®, for which data sheet 3800-3016007, Version 1 dated December, 2007, filed on even date herewith in the accompanying IDS, is hereby incorporated by reference herein in its entirety. Other suitable types of microphones, including other types of capacitive microphones, may be employed.

In still further embodiments, the electromagnetic transducer 25 incorporated into hearing aid 10 is a VKH3391W transducer manufactured by BMH-Tech® of Austria, for which the data sheet filed on even date herewith in the accompanying IDS is hereby incorporated by reference herein in its entirety. Other types of suitable EM transducers may also be used.

FIGS. 3(a) and 3(b) show implantable bone plate or magnetic implant 20 in accordance with FIG. 1(a), where frame 22 has disposed thereon or therein magnetic members 60a and 60b, and where magnetic spacer 50 of hearing aid 10 has magnetic members 55a and 55b spacer disposed therein or thereon. The two magnets 60a and 60b of magnetic implant 20 of FIG. 2(a) permit hearing aid 10 and magnetic spacer 50 to be placed in a single position on patient's skull 70, with respective opposing north and south poles of magnetic members 55a, 60a, 55b and 60b appropriately aligned with respect to one another to permit a sufficient degree of magnetic coupling to be achieved between magnetic spacer 50 and magnetic implant 20 (see also FIG. 3(b)). As shown in FIG. 1(a), magnetic implant 20 is preferably configured to be affixed to skull 70 under patient's skin 75. In one aspect, affixation of magnetic implant 20 to skull 75 is by direct means, such as by screws 15. Other means of attachment known to those skilled in the art are also contemplated, however, such as glue, epoxy, and sutures.

Referring now to FIG. 3(b), there is shown a SOPHONO® ALPHA 1® hearing aid 10 configured to operate in accordance with magnetic implant 20 of FIG. 3(a). As shown, hearing aid 10 of FIG. 3(b) comprises upper housing 111, lower housing 115, magnetic spacer 50, external magnets 55a and 55b disposed within spacer 50, EM transducer diaphragm 45, metal disk 40 connecting EM transducer 25 to spacer 50, programming port/socket 125, program switch 145, and microphone 85. Not shown in FIG. 3(b) are other aspects of the embodiment of hearing aid 10, such as volume control 120, battery compartment 130, battery door 135, battery contacts 140, direct audio input (DAI) 150, and hearing aid circuit board 155 upon which various components are mounted, such as DSP 80.

Continuing to refer to FIGS. 3(a) and 3(b), frame 22 of magnetic implant 20 holds a pair of magnets 60a and 60b that correspond to magnets 55a and 55b included in spacer 50 shown in FIG. 3(b). The south (S) pole and north (N) poles of magnets 55a and 55b, are respectively configured in spacer 50 such that the south pole of magnet 55a is intended to overlie and magnetically couple to the north pole of magnet 60a, and such that the north pole of magnet 55b is intended to overlie and magnetically couple to the south pole of magnet 60b. This arrangement and configuration of magnets 55a, 55b, 60a and 60b is intended permit the magnetic forces required to hold hearing aid 10 onto a patient's head to be spread out or dispersed over a relatively wide surface area of the patient's hair and/or skin 75, and thereby prevent irritation of soreness that might otherwise occur if such magnetic forces were spread out over a smaller or more narrow surface area.

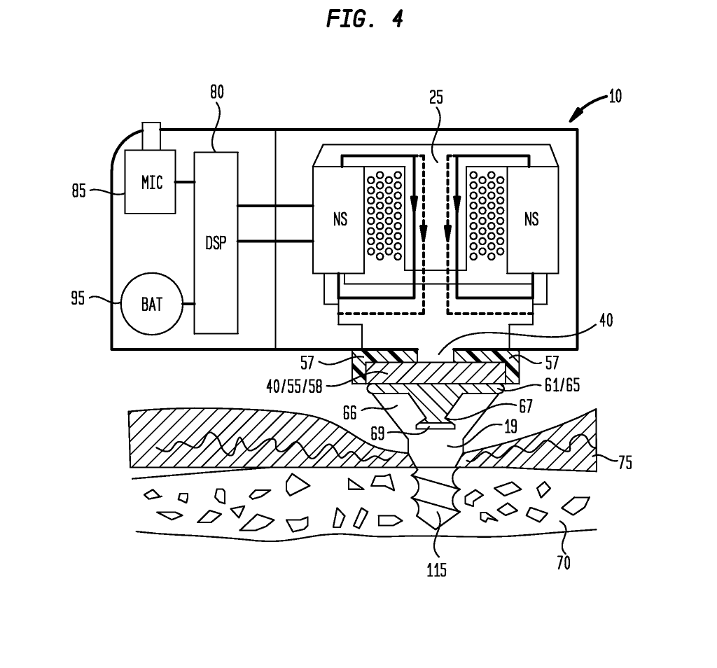

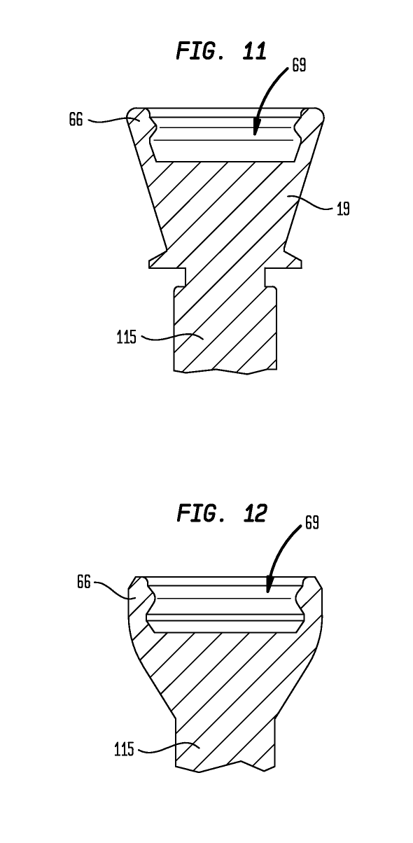

Two principal series of embodiments are described and disclosed herein. In the first such series of embodiments, and referring now to FIGS. 4 through 13, a magnetic hearing aid system and corresponding devices, components, and methods are provided where a bone conduction hearing aid 10 comprises EM transducer 25 and spacer 50, and where spacer 50 comprises at least a first magnetic member 55 or a first ferrous member 58. Spacer 50 is configured to be mechanically and acoustically coupled to EM transducer 25, and to be magnetically and acoustically coupled to an external hearing aid abutment 19 attached to or forming an external portion of a bone screw 115 implanted in a patient's skull 75. At least a second magnetic member 61 (or a second ferrous member 65) is configured to be mounted on or attached to external hearing aid abutment 19. When the second magnetic or ferrous member 61 or 65 is mounted on or attached to external hearing aid abutment 19, and first magnetic or ferrous member 55 or 58 of spacer 50 is placed over and in operable proximity to the mounted or attached second magnetic or ferrous member 61 or 65, first magnetic or ferrous member 55 or 58 and second magnetic or ferrous member 61 or 65 magnetically couple to one another with sufficient magnetic coupling force to secure hearing aid 10 to second magnetic or ferrous member 61 or 65 and external hearing aid abutment 19. At least one of first magnetic or ferrous member 55 or 58 and second magnetic or ferrous member 61 or 65 is magnetic, more about which is said below.

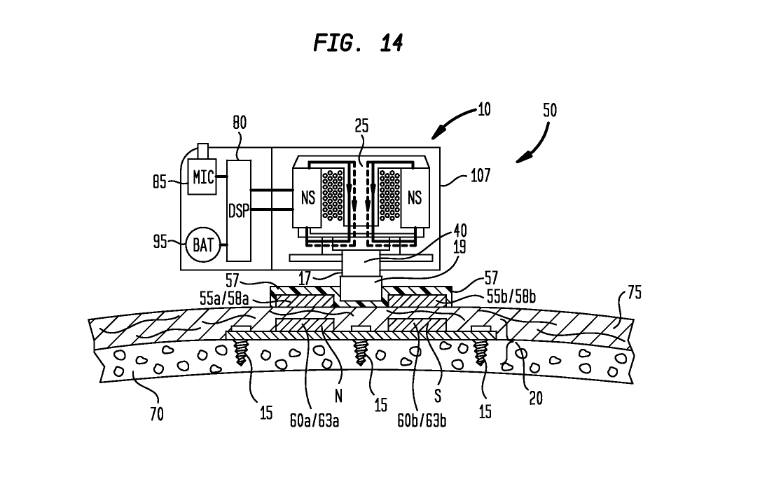

In the second such series of embodiments, and referring to FIGS. 14 through 16, a magnetic hearing aid system and corresponding devices, components, and methods are provided where bone conduction hearing aid 10 comprises EM transducer 25 and attachment member 17 configured to be mechanically and acoustically coupled to EM transducer 25, and where spacer 50 is provided comprising at least first magnetic or ferrous member 55 or 58 and abutment 19 shaped and configured to be mechanically and acoustically coupled to attachment member 17. Magnetic or ferrous implant 20 comprises at least second magnetic or ferrous member 60 or 63, where magnetic or ferrous implant 20 is configured to be implanted beneath patient's skin 75 and affixed or attached to patient's skull 70. First magnetic or ferrous member 55 or 58 of spacer 50 is configured to be magnetically coupled to second magnetic or ferrous member 60 or 63 of magnetic or ferrous implant 20 through patient's skin 75 such that hearing aid 10 and the spacer 50 may be operably secured to patient's skull 75. At least one of first magnetic or ferrous member 55 or 58 and second magnetic or ferrous member 60 or 63 is magnetic.

Referring now to FIGS. 4 through 13, there are shown various embodiments of the first series of embodiments described above comprising spacers 50, abutments 19, and implant 20, any one of which may be either ferrous or magnetic. When spacer 50 is employed in conjunction with abutment 19 as described herein, at least one of first magnetic or ferrous member 55 or 58, member 61 mounted on or attached to abutment 19, a portion of abutment 19, or abutment 19 must be magnetic. When spacer 50 is employed in conjunction with implant 20 as described herein, at least one of first magnetic or ferrous member 55 or 58, a portion of implant 20, implant 20, and/or second magnetic or ferrous member 60 or 63, must be magnetic. Regardless of whether the first magnetic or ferrous member 55 or 58 or the second magnetic or ferrous member 60, 61 or 63 is ferrous or magnetic, or whether both such members are magnetic, sufficient magnetic coupling force must be provided between the first and second magnetic or ferrous members such that hearing aid 10 and spacer 50 are operably secured to patient's skull 75.

By a “ferrous member” we mean a member comprising a metal containing at least some iron. By a “magnetic member” we mean a member comprising a ferromagnetic material that exhibits an attraction to a magnetic field, and that retains its magnetic properties after the magnetic field has been removed.

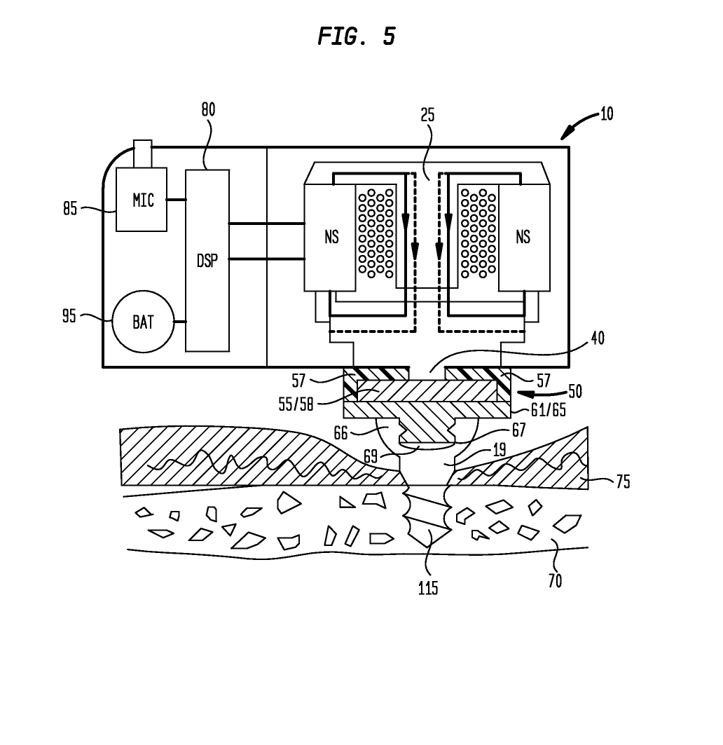

Referring to now to FIGS. 4 and 5, there are shown two different embodiments of hearing aid 10 and corresponding spacer 50 and bone screw 115 comprising first magnetic or ferrous member 55/58 attached to bone screw 115 via abutment 19 having second magnetic or ferrous member 61 attached thereto or secured therein. In the embodiment shown in FIG. 4, at least one or both of first magnetic member 55 or first ferrous member 58 and second magnetic member 61 or second ferrous member 65 is magnetic so as to provide a sufficient amount of magnetic coupling force between first magnetic or ferrous member 55/58 and second magnetic or ferrous member 61/65.

In one such embodiment, magnetic or ferrous member 55/58 is magnetic and comprises, by way of example, a rare earth magnetic material such neodymium, and second ferrous member 65 comprises a ferrous metal that permits a sufficient degree or amount of magnetic coupling to occur between members 55 and 65.

In another such embodiment, magnetic or ferrous member 55/58 is a ferrous member 58 comprising, by way of example, a ferrous metal such as steel containing iron, and second magnetic member 61 is magnetic and comprises, by way of example, a rare earth magnetic material such neodymium that permits a sufficient degree or amount of magnetic coupling to occur between members 58 and 61.

In yet another embodiment, magnetic or ferrous member 55/58 is a magnetic member 55 comprising, by way of example, a rare earth magnetic material such as neodymium, and second magnetic member 61 is magnetic and also comprises, by way of example, a rare earth magnetic material such as neodymium that permits a sufficient degree or amount of magnetic coupling to occur between members 55 and 61.

Referring still to FIG. 4, it will be seen that second magnetic or ferrous member 61/65 attaches to or is mounted in or on abutment 19, which comprises outer shoulders 66 and recess 69 into which protruding portion 67 of second magnetic or ferrous member 61/65 is configured to fit. Similarly, in FIG. 5 second magnetic or ferrous member 61/65 attaches to or is mounted in or on abutment 19, which comprises outer shoulders 66 and recess 69 into which protruding portion 67 of second magnetic or ferrous member 61/65 is configured to fit. Note that outer shoulders 66 of abutment 19 in FIG. 4 differ from outer shoulder 66 of abutment 19 in FIG. 5. Outer shoulders 66 in FIG. 4 have an outer diameter that reduces in diameter in linear fashion towards bone screw 115. Contrariwise, outer shoulders 66 in FIG. 5 are convexly shaped.

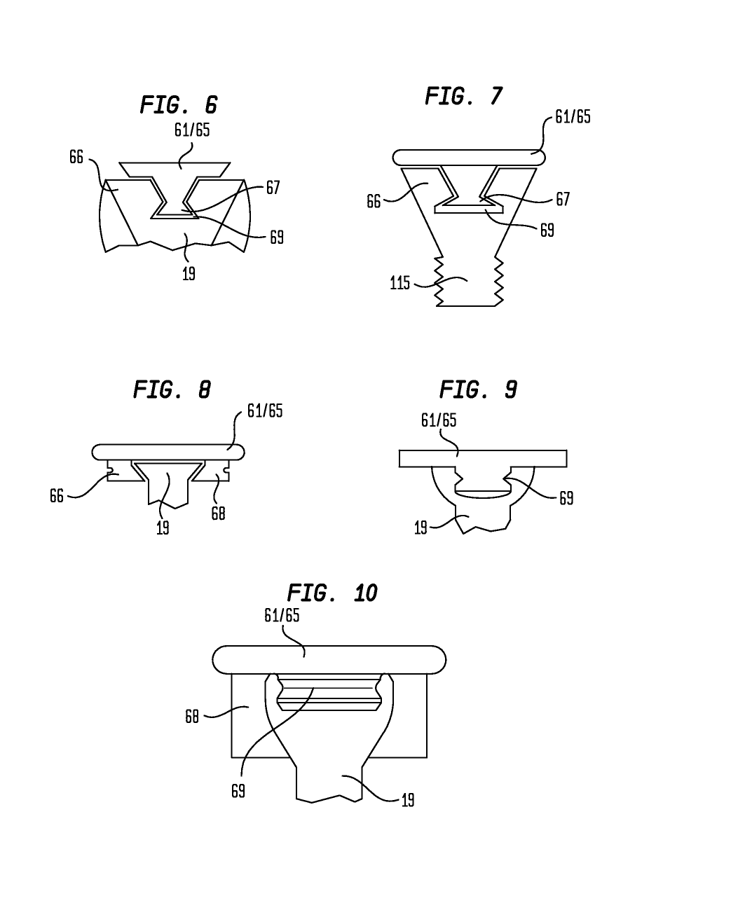

FIGS. 6 through 12 show various embodiments of abutment 19 and bone screw 115, and of corresponding second magnetic or ferrous member 61/65. In FIGS. 6 and 7, there is shown one embodiment of an upper portion of abutment 19 with recess 69 formed therein that is configured to accept one embodiment of second magnetic or ferrous member 61/65 therein. FIG. 8 shows another embodiment of second magnetic or ferrous member 61/65, which is configured to fit upon and engage outer shoulders 66 of abutment 19, and which includes shoulder engagement member 68 attached thereto or forming a portion thereof. FIG. 9 shows another embodiment of an upper portion of abutment 19 having convex-shaped outer shoulders 66 and recess 69 formed therein. Recess 69 is configured to accept second magnetic or ferrous member 61/65 therein. FIG. 10 shows yet another embodiment of second magnetic or ferrous member 61/65, which is configured to fit upon and engage convexly-shaped outer shoulders 66 of abutment 19, and which includes shoulder engagement member 68 attached thereto or forming a portion thereof. Thus, according to various embodiments, second magnetic or ferrous members 61/65 are configured to be attached to or mounted on the outer shoulders of abutment 19, and second magnetic or ferrous members 61/65 are therefore not limited to being configured only to be mounted in or attached to recesses 69 of abutment 19.

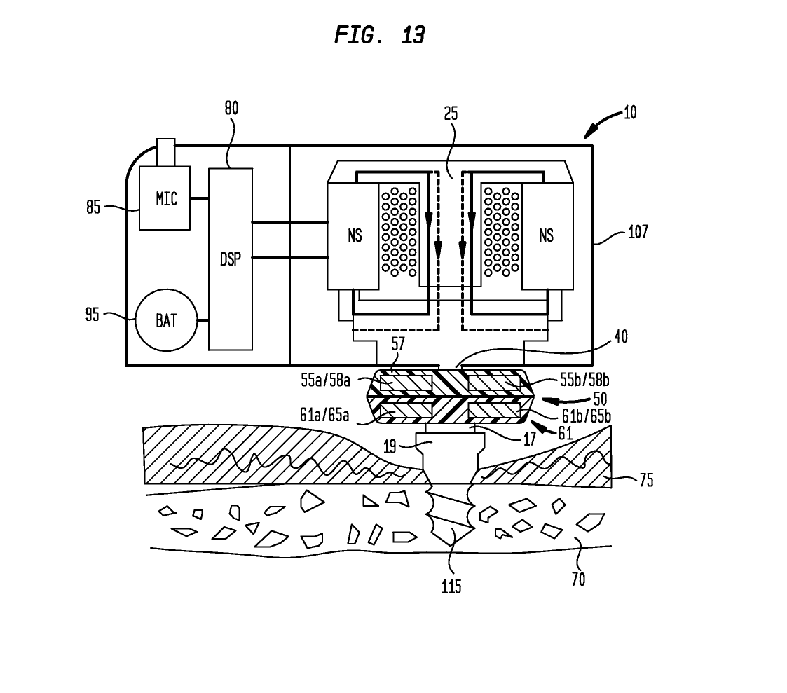

Referring now to FIG. 13, there is shown another embodiment of spacer 50 comprising first magnetic or ferrous member 55/58 that is configured to couple magnetically to second magnetic or ferrous member 61/65. More particularly, first magnetic or ferrous members 55a/58a and 55b/58b are configured to couple magnetically with corresponding second magnetic or ferrous members 61a/65a and 61 b/65b, which are mounted on or attached to abutment 19 of bone screw 115 via attachment member 17, which, by way of example, may assume a shape similar to that of second magnetic or ferrous member 61/65 shown in FIGS. 4 through 10. In the embodiment illustrated in FIG. 13, a standard prior art ALPHA1 hearing aid 10 and corresponding magnetic spacer 50 may be magnetically coupled to several different types of conventional prior art bone screws 115 via abutment engagement member 17 and second magnetic or ferrous members 61a/65a and 61b/65b.

As described above, FIGS. 14 through 16 show a second series of embodiments of a magnetic hearing aid system and corresponding devices, components, and methods, where bone conduction hearing aid 10 and spacer 50 may be operably secured to patient's skull 75 and implant 20 through suitable magnetic coupling forces.

FIG. 14 shows one embodiment of such a system where a conventional (e.g., non-magnetic) hearing aid 10 is mechanically coupled to attachment member 17, which in turn is configured to fit into a recess in or the outer shoulders of abutment 19, which in turn is attached to spacer 50. Note that recesses 69 and outer shoulders 66 of abutment 19 may assume any of the shapes shown in FIGS. 6 through 12, or any other suitable shapes. In FIG. 14, first magnetic or ferrous members 55a/58a and 55b/58b of space 50 are configured to couple magnetically to corresponding second magnetic or ferrous members 60a/63a and 60b/63b disposed in or on implant 20, which is affixed to patient's skull 75. According to FIG. 14, and in one embodiment, a conventional non-magnetic hearing aid 10 may thus be magnetically coupled to a SOPHONO magnetic implant 20 via spacer 50. Note that other embodiments are contemplated, including, but not limited to, embodiments where implants 20 are ferrous but not magnetic, and where first magnetic or ferrous members are ferrous but not magnetic.

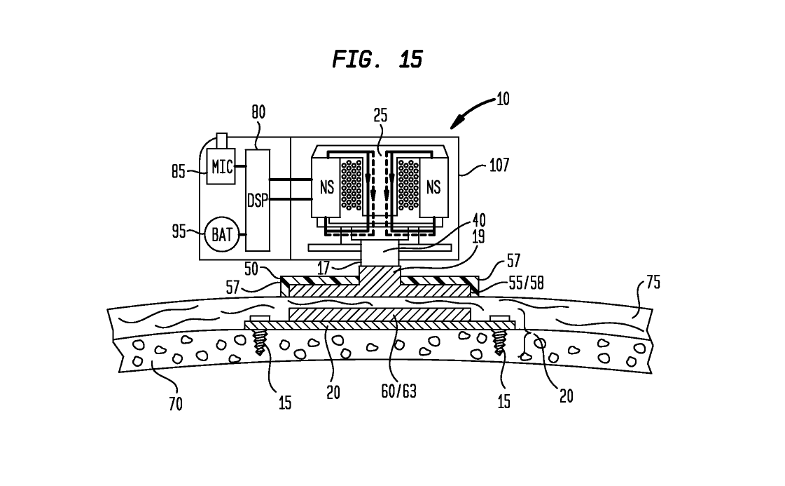

Referring now to FIG. 15, there is shown one embodiment where a conventional (e.g., non-magnetic) hearing aid 10 may be mechanically coupled to attachment member 17, which is configured to fit into a recess in or onto outer shoulders 66 of abutment 19, which in turn is attached to spacer 50. Note that recesses 69 and outer shoulders 66 of abutment 19 may assume any of the shapes or configurations shown in FIGS. 6 through 12, or any other suitable shapes or configurations. In FIG. 14, first magnetic or ferrous members 55a/58a and 55b/58b of space 50 are configured to couple magnetically to corresponding second magnetic or ferrous members 60a/63a and 60b/63b disposed in or on implant 20, which is affixed to patient's skull 75. Note that implant 20 may ferrous but not magnetic, and first magnetic or ferrous members 55/58 may be ferrous but not magnetic. Implant 20 and/or magnetic or ferrous members 55/58 may also be magnetic.

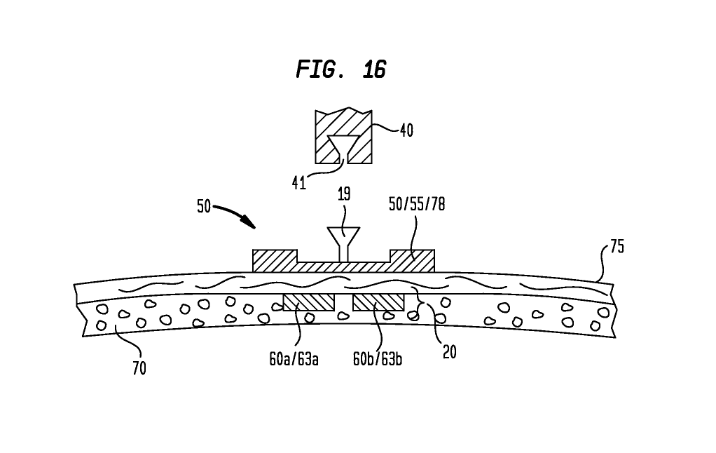

FIG. 16 shows another embodiment where a conventional (e.g., non-magnetic) hearing aid 10 may be mechanically coupled to abutment 19 via attachment member 17 and corresponding recess 41. Attachment member 17 is attached to hearing aid 10 directly, or in another embodiment to spacer 50. Note that the recesses and outer shoulders of abutment 19 may assume any of the shapes or configurations shown in FIGS. 6 through 12, or any other suitable shapes or configurations. In FIG. 16, first magnetic or ferrous member 55/58 is configured to couple magnetically to corresponding second magnetic or ferrous members 60a/63a and 60b/63b disposed in or on implant 20, which is affixed to patient's skull 75. According to FIG. 16, and in one embodiment, a conventional non-magnetic hearing aid 10 may thus be magnetically coupled to a SOPHONO magnetic implant 20 via spacer 50. Note that other embodiments are contemplated, however, such as implants 20 that are ferrous but not magnetic, and first magnetic or ferrous members 50/55 that are ferrous but not magnetic.

Referring to FIGS. 4 through 16, those skilled in the art will now appreciate that many different combinations, permutations and configurations of spacers 50, implants 20, magnetic or ferrous members 55, 58, 60, 61, 63 and 65, abutments 19, attachment members 17, recesses 69, outer shoulders 66, magnetic adhesion or pull forces may be employed to arrive at a hearing aid 10 that can be suitably magnetically coupled to a patient's skull. For example, in some embodiments first magnetic or ferrous member 55/58 is magnetic and second magnetic or ferrous member 61/65 or 60/63 is also magnetic. In other embodiments, first magnetic or ferrous member 55/58 is ferrous and second magnetic or ferrous member 61/65 or 60/63 is magnetic. In further embodiments, first magnetic or ferrous member 55/58 is magnetic and second magnetic or ferrous member 61/65 or 60/63 is ferrous. External hearing aid abutment 19 may have a recess 69 configured to receive at least a portion of second magnetic or ferrous member 61/65 therein or thereon. A portion of second magnetic or ferrous member 61/65 may be configured to be disposed over or attached to outer shoulder 66 of external hearing aid abutment 19. At least one of first and second ferrous or magnetic members 55/58 and/or 61/65 or 60/63 may comprise a rare earth magnetic material. First and second ferrous or magnetic members 55/58, 61/65, and/or 60/63 may have a thicknesses ranging between about 1 mm and about 4 mm. The adhesion force between first magnetic or ferrous member 55/58 and second magnetic or ferrous member 62/65 or 60/63 may range between about 0.5 Newtons and about 3 Newtons, or between about 1 Newton and about 2.5 Newtons.

Continuing to refer to FIGS. 4 through 16, the various embodiments described and disclosed herein include methods, such as methods where a patient or healthcare provider places first magnetic or ferrous member 55/58 of spacer 50 and hearing aid 10 over and in operable proximity to mounted or attached second magnetic or ferrous member 61/65, and magnetically coupling first magnetic or ferrous member 55/58 to second magnetic or ferrous member 61/65 with sufficient magnetic coupling force to secure hearing aid 10 to patient's skull 75. Such methods further include a patient or healthcare provider placing first magnetic or ferrous member 55/58 of spacer 50 and hearing aid 10 over and in operable proximity to an implanted second magnetic or ferrous member 60/63, and magnetically coupling first magnetic or ferrous member 55/58 to second magnetic or ferrous member 60/63 with sufficient magnetic coupling force to secure hearing aid 10 to patient's skull 75.

See also, for example, U.S. Pat. No. 7,021,676 to Westerkull entitled “Connector System,” U.S. Pat. No. 7,065,223 to Westerkull entitled “Hearing-Aid Interconnection System,” and U.S. Design Pat. No. D596,925 S to Hedstrom et al., which disclose bone screws, abutments and hearing aids that may be modified in accordance with the teachings and disclosure made herein, each of which is hereby incorporated by reference herein, each in its respective entirety.

The above-described embodiments should be considered as examples of the present invention, rather than as limiting the scope of the invention. In addition to the foregoing embodiments of the invention, review of the detailed description and accompanying drawings will show that there are other embodiments of the present invention. Accordingly, many combinations, permutations, variations and modifications of the foregoing embodiments of the present invention not set forth explicitly herein will nevertheless fall within the scope of the present invention.

We claim:

1. A magnetic hearing aid system, comprising:
a bone conduction hearing aid comprising an electromagnetic (“EM”) transducer and a spacer comprising at least a first magnetic or ferrous member, the spacer being configured to be mechanically and acoustically coupled to the EM transducer, and to be magnetically and acoustically coupled to an external hearing aid abutment attached to or forming an external portion of a bone screw implanted in a patient's skull, and
at least a second magnetic or ferrous member configured to be mounted on or attached to the external hearing aid abutment;
wherein when the second magnetic or ferrous member is mounted on or attached to the external hearing aid abutment, and the first magnetic or ferrous member of the spacer is placed over and in operable proximity to the mounted or attached second magnetic or ferrous member, the first magnetic or ferrous member and the second magnetic or ferrous member magnetically couple to one another with sufficient magnetic coupling force to secure the hearing aid to the second magnetic or ferrous member and the external hearing aid abutment, and further wherein at least one of the first magnetic or ferrous member and the second magnetic or ferrous member is magnetic.

2. The magnetic hearing aid system of claim 1, wherein the first magnetic or ferrous member is magnetic and the second magnetic or ferrous member is also magnetic.

3. The magnetic hearing aid system of claim 1, wherein the first magnetic or ferrous member is ferrous and the second magnetic or ferrous member is magnetic.

4. The magnetic hearing aid system of claim 1, wherein the first magnetic or ferrous member is magnetic and the second magnetic or ferrous member is ferrous.

5. The magnetic hearing aid system of claim 1, wherein the external hearing aid abutment has a recess configured to receive at least a portion of the second magnetic or ferrous member therein or thereon.

6. The magnetic hearing aid system of claim 1, wherein a portion of the second magnetic or ferrous member is configured to be disposed over or attached to an outer shoulder of the external hearing aid abutment.

7. The magnetic hearing aid system of claim 1, wherein at least one of the first and second ferrous or magnetic members comprises a rare earth magnetic material.

8. The magnetic hearing aid system of claim 1, wherein at least one of the first and second ferrous or magnetic members has a thicknesses ranging between about 1 mm and about 4 mm.

9. The magnetic hearing aid system of claim 1, wherein an adhesion force between the first magnetic or ferrous member and the second magnetic or ferrous member ranges between about 0.5 Newtons and about 3 Newtons.

10. The magnetic hearing aid system of claim 1, wherein an adhesion force of between the first magnetic or ferrous member and the second magnetic or ferrous member ranges between about 1 Newton and about 2.5 Newtons.

11. A second magnetic or ferrous member configured to be mounted on or attached to an external hearing aid abutment, the hearing aid abutment being configured to be attached to or form an external portion of a bone screw implanted in a patient's skull, the bone screw and hearing aid abutment being configured to operate in conjunction with a bone conduction hearing aid comprising an electromagnetic (“EM”) transducer and a spacer comprising at least a first magnetic or ferrous member, the spacer being configured to be mechanically and acoustically coupled to the EM transducer, and to be magnetically and acoustically coupled to the second magnetic or ferrous member and the external hearing aid abutment, wherein when the second magnetic or ferrous member is mounted on or attached to the external hearing aid abutment, and the first magnetic or ferrous of the spacer and hearing aid is placed over and in operable proximity to the mounted or attached second magnetic or ferrous member, the first magnetic or ferrous member and the second magnetic or ferrous member magnetically couple to one another with sufficient magnetic coupling force to secure the hearing aid to the second magnetic or ferrous member and the external hearing aid abutment, and further wherein at least one of the first magnetic or ferrous member and the second magnetic or ferrous member is magnetic.

12. The second magnetic or ferrous member of claim 11, wherein the second magnetic or ferrous member is magnetic.

13. The second magnetic or ferrous member of claim 11, wherein the second magnetic or ferrous member is ferrous.

14. The second magnetic or ferrous member of claim 11, wherein the second magnetic or ferrous member is configured to be received in a recess disposed in the external hearing aid abutment.

15. The second magnetic or ferrous member of claim 11, wherein a portion of the second magnetic or ferrous member is configured to be disposed over or attached to an outer shoulder of the external hearing aid abutment.

16. The second magnetic or ferrous member of claim 11, wherein the second ferrous or magnetic member comprises a rare earth magnetic material.

17. The second magnetic or ferrous member of claim 11, wherein the second ferrous or magnetic member has a thickness ranging between about 1 mm and about 4 mm.

18. The second magnetic or ferrous member of claim 11, wherein an adhesion force between the first magnetic or ferrous member and the second magnetic or ferrous member ranges between about 0.5 Newtons and about 3 Newtons.

19. The second magnetic or ferrous member of claim 11, wherein an adhesion force of between the first magnetic or ferrous member and the second magnetic or ferrous member ranges between about 1 Newton and about 2.5 Newtons.

20. A method of magnetically coupling a first magnetic or ferrous member to a second magnetic or ferrous member, the second magnetic or ferrous member being configured to be mounted on or attached to an external hearing aid abutment, the hearing aid abutment being configured to be attached to or form an external portion of a bone screw implanted in a patient's skull, the bone screw and hearing aid abutment being configured to operate in conjunction with a bone conduction hearing aid comprising an electromagnetic (“EM”) transducer and a spacer comprising at least the first magnetic or ferrous member, the spacer being configured to be mechanically and acoustically coupled to the EM transducer, and to be magnetically and acoustically coupled to the second magnetic or ferrous member and the external hearing aid abutment, at least one of the first magnetic or ferrous member and the second magnetic or ferrous member being magnetic, the method comprising:
a patient or healthcare provider placing the first magnetic or ferrous member of the spacer and hearing aid over and in operable proximity to the mounted or attached second magnetic or ferrous member, and
magnetically coupling the first magnetic or ferrous member to the second magnetic or ferrous member with sufficient magnetic coupling force to secure the hearing aid to the patient's skull.

21. The method of claim 20, further comprising the patient or healthcare provider mounting or attaching the second magnetic or ferrous member to the external hearing aid abutment.

22. A magnetic hearing aid system, comprising:
a bone conduction hearing aid comprising an electromagnetic (“EM”) transducer and an attachment member configured to be mechanically and acoustically coupled to the EM transducer;
a spacer comprising at least a first magnetic or ferrous member and an abutment shaped and configured to be mechanically and acoustically coupled to the attachment member, and
a magnetic or ferrous implant comprising at least a second magnetic or ferrous member, the magnetic or ferrous implant being configured to be implanted beneath a patient's skin and affixed or attached to the patient's skull;
wherein the first magnetic or ferrous member of the spacer is configured to be magnetically coupled to the second magnetic or ferrous member of the magnetic or ferrous implant through the patient's skin such that the hearing aid and the magnetic spacer may be operably secured to the patient's skull, and further wherein at least one of the first magnetic or ferrous member and the second magnetic or ferrous member is magnetic.

23. The magnetic hearing aid system of claim 22, wherein the first magnetic or ferrous member is magnetic and the second magnetic or ferrous member is also magnetic.

24. The magnetic hearing aid system of claim 22, wherein the first magnetic or ferrous member is ferrous and the second magnetic or ferrous member is magnetic.

25. The magnetic hearing aid system of claim 22, wherein the first magnetic or ferrous member is magnetic and the second magnetic or ferrous member is ferrous.

26. The magnetic hearing aid system of claim 22, wherein at least one of the first and second ferrous or magnetic members comprises a rare earth magnetic material.

27. The magnetic hearing aid system of claim 22, wherein at least one of the first and second ferrous or magnetic members has a thicknesses ranging between about 1 mm and about 4 mm.

28. The magnetic hearing aid system of claim 22, wherein an adhesion force between the first magnetic or ferrous member and the second magnetic or ferrous member ranges between about 0.5 Newtons and about 3 Newtons.

29. The magnetic hearing aid system of claim 22, wherein an adhesion force of between the first magnetic or ferrous member and the second magnetic or ferrous member ranges between about 1 Newton and about 2.5 Newtons.

30. A spacer configured for use in a hearing aid system comprising a bone conduction hearing aid comprising an electromagnetic (“EM”) transducer, an attachment member configured to be mechanically and acoustically coupled to the EM transducer, and a magnetic or ferrous implant comprising at least a second magnetic or ferrous member, the magnetic or ferrous implant being configured to be implanted beneath a patient's skin and affixed or attached to the patient's skull, wherein the spacer comprises at least a first magnetic or ferrous member and an abutment shaped and configured to be mechanically and acoustically coupled to the attachment member, the first magnetic or ferrous member of the spacer being configured to be magnetically coupled to the second magnetic or ferrous member of the magnetic or ferrous implant through the patient's skin such that the hearing aid and the spacer may be operably secured to the patient's skull, and further wherein at least one of the first magnetic or ferrous member and the second magnetic or ferrous member is magnetic.

31. The spacer of claim 30, wherein the first magnetic or ferrous member is magnetic.

32. The spacer of claim 30, wherein the first magnetic or ferrous member is ferrous.

33. The spacer of claim 30, wherein the first ferrous or magnetic member comprises a rare earth magnetic material.

34. The spacer of claim 30, wherein the first ferrous or magnetic member has a thickness ranging between about 1 mm and about 4 mm.

35. The spacer of claim 30, wherein an adhesion force between the first magnetic or ferrous member and the second magnetic or ferrous member ranges between about 0.5 Newtons and about 3 Newtons.

36. The spacer of claim 30, wherein an adhesion force of between the first magnetic or ferrous member and the second magnetic or ferrous member ranges between about 1 Newton and about 2.5 Newtons.

37. A method of magnetically coupling a first magnetic or ferrous member to a second magnetic or ferrous member, the first magnetic or ferrous member forming a portion of a spacer comprising an abutment, the spacer being configured for use in a hearing aid system comprising a bone conduction hearing aid and a magnetic or ferrous implant comprising at least a second magnetic or ferrous member, the hearing aid comprising an electromagnetic (“EM”) transducer and an attachment member configured to be mechanically and acoustically coupled to the EM transducer and the abutment, the magnetic or ferrous implant being configured to be implanted beneath a patient's skin and affixed or attached to the patient's skull, at least one of the first magnetic or ferrous member and the second magnetic or ferrous member being magnetic, the method comprising:
a patient or healthcare provider placing the first magnetic or ferrous member of the spacer and hearing aid over and in operable proximity to the implanted second magnetic or ferrous member, and
magnetically coupling the first magnetic or ferrous member to the second magnetic or ferrous member with sufficient magnetic coupling force to secure the hearing aid to the patient's skull.
